(19) United States
(12) Patent Application Publication
(10) Pub. No.: US 2023/0000517 A1
(43) Pub. Date: Jan. 5, 2023

(54) Method and apparatus for oral hygiene

(71) Applicant: Tonsil Tech, Inc., Redmond, OR (US)

(72) Inventors: Jessalyn Imdieke, Molalla, OR (US); Sydney Quinton-Cox, Redmond, OR (US); Daniel Forbes, Redmond, OR (US)

(21) Appl. No.: 17/481,908

(22) Filed: Sep. 22, 2021

Related U.S. Application Data
(60) Provisional application No. 63/216,717, filed on Jun. 30, 2021.

(51) Int. Cl. A61B 17/3205; A61B 17/26; A61B 17/3207

(52) U.S. Cl. CPC A61B 17/32056; A61B 17/26; A61B 17/320708

(57) ABSTRACT

An oral hygiene device having a main body including a first end and a second end, with a main body extending therebetween. At least one end has an extraction tip. The main body may be composed of a flexible material. The device may be used for removing tonsil stones.

### CROSS-REFERENCE TO RELATED APPLICATIONS

This application claims the benefit of priority of U.S. Provisional Patent Application Ser. No. 63/216,717, filed on Jun. 30, 2021, and entitled “TONSIL STONE EXTRACTOR”, which is incorporated by reference herein in its entirety.

### FIELD OF THE INVENTION

The present invention generally relates to the field of general wellness oral hygiene. In particular, the present invention is directed to an oral hygiene device for removing tonsil stones.

### BACKGROUND

Varying complex oral healthcare issues exist.

Some tonsils naturally have crypts. These crypts have varying sizes in the initial opening and in the cavity itself which can collect oral debris. Tonsil stones are a type of oral debris that form in the crypts of tonsils. Tonsil stones are the calcification of bacteria, food, mucus, and other oral debris that has accumulated in the crypts of the tonsils.

### SUMMARY OF THE DISCLOSURE

An oral hygiene device for removing tonsil stones comprising a main body wherein the main body includes a first end and second end wherein the first end includes a handle and wherein second end includes an extraction tip and wherein the main body is composed of a flexible material.

In an aspect there is an aperture in the extraction tip and the aperture is configured to hold a tonsil stone wherein the aperture is teardrop shaped and wherein the aperture has a first side measuring less than 10 mm.

In an aspect a handle portion comprises a textured length for producing a non-slip grip.

In an aspect a neck between the extraction tip and handle may be cylindrical or ovular in shape and may connect to the base of the extraction tip at the smallest diameter and gradually taper to match the diameter of the handle.

In an aspect an oral hygiene device comprises a main body extending between a handle and an extraction tip.

In an aspect an aperture in the extraction tip has a first cross-sectional dimension measuring less than 10 mm.

In an aspect a handle portion comprises a textured length for producing a non-slip grip.

In an aspect there is a neck between the extraction tip and handle.

In an aspect an oral hygiene device for removing tonsil stones comprises a main body extending between a handle and an extraction tip, wherein one or both of the main body and extraction tip are comprised of a stimuli-responsive material such that a geometry of the device is customizable using one or more stimuli selected from the group consisting of light, temperature, pH, humidity, electricity, and magnetism.

In an aspect an aperture in the extraction tip has a length, width, or cross-sectional dimension at its widest or longest point that is less than 10 mm.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

The present invention is illustrated by way of example and not limitation in the figures of the accompanying drawings in which like references indicate similar elements. For the purpose of illustrating the invention, the drawings show aspects of one or more embodiments of the invention. However, it should be understood that the present invention is not limited to the precise arrangements and instrumentalities shown in the drawings, wherein:

FIG. 1 is a perspective view of a tonsil stone extractor device.

FIG. 2A illustrates a shape of a tip of the device.

FIG. 2B is a perspective view of the tip of the device.

FIGS. 3A-3C are front views illustrating varying size tips of the device.

FIG. 4A is a front view of a handle of the device.

FIG. 4B is a side view of the handle of the device.

FIG. 4C is a perspective view of the handle of the device.

FIG. 5A is a front view of the device.

FIG. 5B is a side view of the device.

FIG. 5C is another perspective view of the device with lines indicating flexibility.

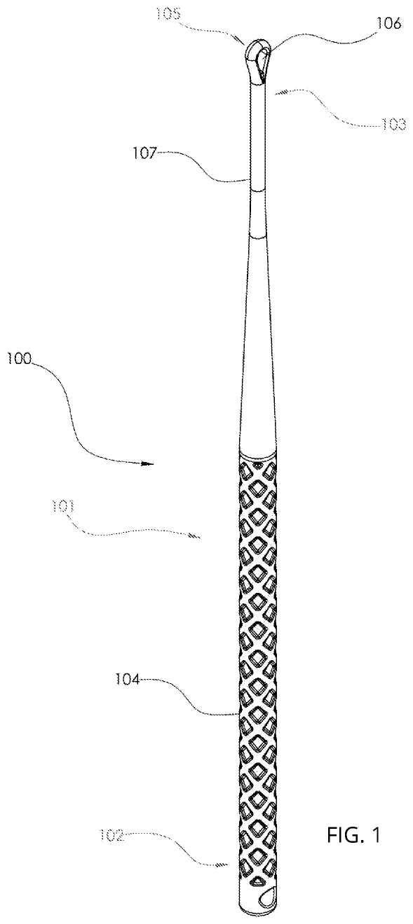

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

Various embodiments and aspects of the inventions will be described with reference to details discussed below, and the accompanying drawings will illustrate the various embodiments. The following description and drawings are illustrative of the invention and are not to be construed as limiting the invention. Numerous specific details are described to provide a thorough understanding of various embodiments of the present invention. However, in certain instances, well-known or conventional details are not described in order to provide a concise discussion of embodiments of the present inventions.

An oral hygiene device 100 according to one or more embodiments may be used for removing tonsil stones. The device 100 may feature a main body 101 wherein the main body 101 includes a first end 102 and a second end 103 wherein the first end 102 may include a handle 104 and wherein the second end 103 may include an extraction tip 105 and wherein the main body 101 may be composed of a flexible material.

The device 100 is configured to remove tonsil stones in the palatine tonsils. Further, the device 100 is one that removes and or exposes tonsil stones without the use of suction. In one or more embodiments the device 100 is a manual device 100 that requires only rotation or application of manual (for example downward or inward) pressure, angled movement in the manner of a wedge or combinations thereof to safely and easily remove or expose a tonsil stone.

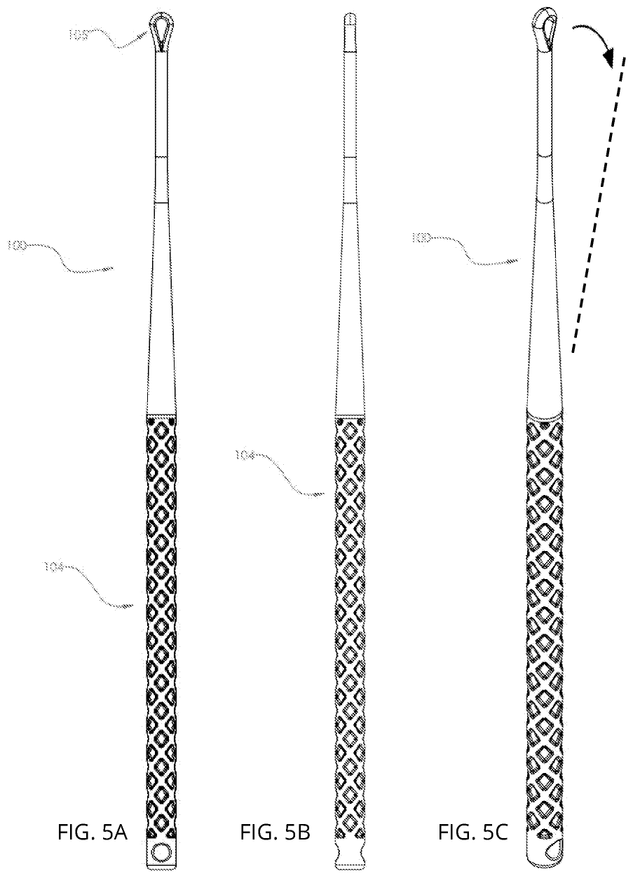

Flexibility, as used throughout this disclosure refers to a material's ability to move or bend when force is applied without breaking. In this context, flexibility is used to describe the device's 100 ability to be deformed when pressure is applied from a user's fingers or mouth. A flexible material allows the device 100 to deform such that if excess force is applied during use, the material will absorb the force and bend, rather than transfer the force to the user's tonsil. This absorption of force by the material and device 100 may prevent local tissue injury and provide more comfort to the user of the device 100. Depending on the properties of the material, a flexible material used in the extraction tip 105 may also allow the extraction tip 105 to deform and compress the aperture 106, trapping a tonsil stone using applied force rather than the tonsil stone being held in place using friction. The main areas where flexibility is utilized are in the neck 107 to the extraction tip 105, to absorb extra force, and in the extraction tip 105, to trap a tonsil stone. As illustrated in FIG. 5C, the device 100 may have areas of flexibility extending a length from and including the neck 107 to the extraction tip 105 region. Flexibility, as used throughout this disclosure, also describes the ability of the flexible material to hold its shape when it is intentionally bent. The device 100 may be bent at an angle, hold the bent shape, and then be bent back to the original shape by the user. The ability for the device 100 to retain a customized bent shape is important for users to use the device 100 at different angles within their oral cavity to approach the tonsil crypts from different directions, therefore accessing tonsil stones that were previously inaccessible. Since people have different shaped tonsils which have different shaped and angled tonsil crypts, having the user manipulate the shape of the device 100 may allow for more successful extraction of the tonsil stones.

Other methods to achieve the shape customization include using a stimuli-responsive material using one or more stimuli selected from the group consisting of light, temperature, pH, humidity, electricity, and magnetism. Using a stimuli responsive material allows the user to customize the device 100 to meet the specific geometric requirements of their oral cavity and tonsillar crypt.

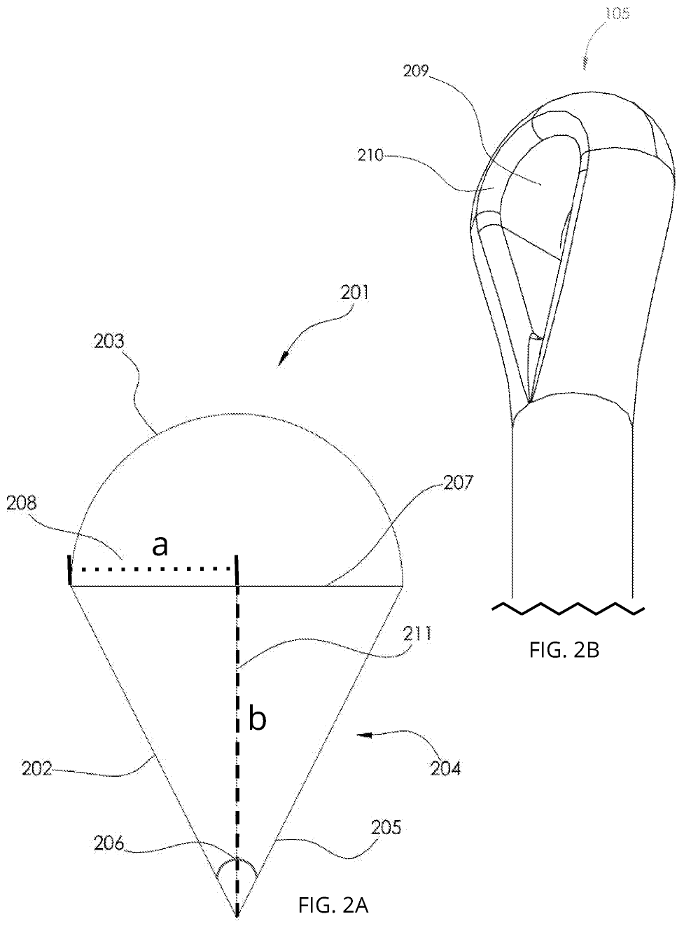

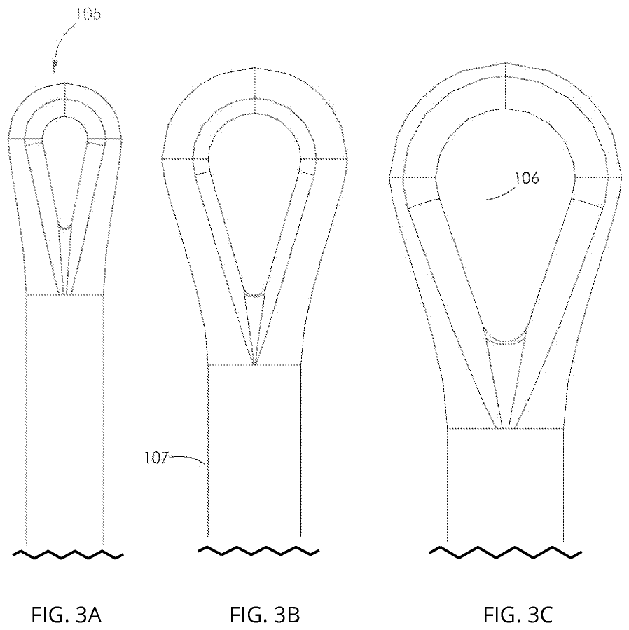

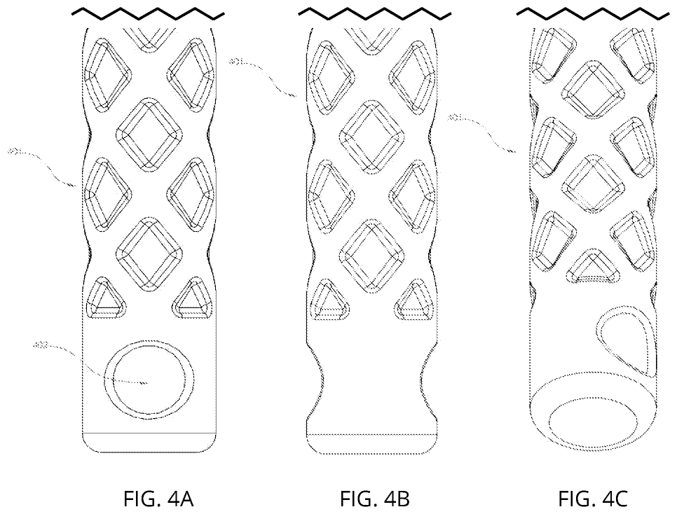

As illustrated in FIGS. 2A-2B and FIGS. 3A-3C, the device 100 may feature an aperture 106 in the extraction tip 105 wherein the aperture 106 is configured to hold a tonsil stone. The aperture 106 may be teardrop shaped 201 and the aperture 106 may have a first side 202 measuring less than 10 mm.

The teardrop shaped 201 aperture 106 may be described as comprised of a semicircular 203 aperture 106 connected to a triangular 204 aperture 106 which together form the overall teardrop shaped 201 aperture 106, as illustrated in FIG. 2A. In one or more embodiments, the combination of the semicircular 203 aperture 106 and triangular 204 aperture 106 form a teardrop shaped 201 aperture 106 which may be longer than it is wide. For example, the triangular 204 shaped aperture 106 may be an isosceles triangle 204 in shape wherein a diameter of the semicircular 203 aperture 106 is substantially equal to a shortest side length of the teardrop shaped 201 aperture 106 defined by the triangular 204 aperture 106. In further detail, the teardrop shaped 201 aperture 106 may be described as having a first side 202 length provided by one side length or leg of the triangular 204 aperture 106. A second side 205 length may be considered an opposing side length or leg of the triangular 204 aperture 106. The first side 202 length and second side 205 length may be of equal length and meet to form the acute angle 206 of the teardrop shaped 201 aperture 106. The acute angle 206 of the teardrop shaped 201 aperture 106 may also be the narrowest part of the teardrop shaped 201 aperture 106 and may point towards the main body 101 of the device 100 or otherwise be adjacent to the main body 101. Terminal ends of the first side 202 length and second side 205 length which extend away from the main body 101 may provide a maximum width 207 to the teardrop shape 201. This may be considered a base of the triangular shape. This maximum width 207 may also be twice as long as a radius 208 of the semicircle 203. An arcuate length provided by a curved section of the semicircle aperture 106 provides a rounded distal end to the teardrop shaped 201 aperture 106.

An overall size of the teardrop shaped 201 aperture 106 may be described by a ratio between the radius 208 of the semicircle described above and the height 211 at the line of symmetry of the triangle 204. For clarity, in FIG. 2A the dotted line labeled “a” represents the radius of the semicircle, and the dashed line labeled “b” represents the height 211 at the meridian. The ratio of “a” to “b” may be in the range of 1:0.25 to 1:10. In an embodiment the ratio is in the range of 1:2 to 1:5 as illustrated in FIG. 2A. In an embodiment, the aperture 106 shape may include but is not limited to teardrop, a symmetrical teardrop, an asymmetric teardrop, circular, triangular, rectangular, oblong, oval shape, or combination of.

A narrowing geometry in the extraction tip 105 created by the teardrop shape 201 may aid in retaining the tonsil stone by acting as a “wedge.” The increased surface area created by the teardrop shape 201, as opposed to a circular shape, provides more surface area for the tonsil stone to interact with, increasing the frictional forces on the stone, which may further contribute to holding the stone in place.

A tonsil stone may be further held in place within the extraction tip 105 by the depth 209 of the aperture 106. The depth 209 of the aperture 106 may create what is referred to in this disclosure as a platform 209, such that a tonsil stone can be held in place on the platform 209. The platform 209 may include internal geometric features for increased tonsil stone grip using frictional force. These geometric features may include but are not limited to texture, protruding or embossed, or curvature. The curvature may be conceptualized as the internal platform being concave, such that the outside edges around the aperture 106 are higher than the inside center of aperture 106. The added texture or curvature may provide additional surface area to the platform 209 for the tonsil stone to interact with, increasing the frictional forces on the stone, which may further hold it in place. Depending on the flexibility of the material used to create the extraction tip 105, the sides of the extraction tip 105 may compress with applied pressure to trap and grip a tonsil stone using applied force. There may also exist a combination of both internal protruding geometry and extraction tip 105 flexibility to maximize the grip on the tonsil stone such that when the extraction tip 105 is compressed, internal protruding geometry may be pressed into the stone to lock it in place.

The extraction tip 105 may be inserted into the tonsil crypt. As opposed to applying external pressure directly to the tonsil to express a tonsil stone, potentially damaging the tonsil tissue, inserting the extraction tip 105 directly into the crypt of the tonsil to capture the stone is a safer and more effective method of removing the stone since some stones are wedged in place within a tonsil such that no amount of applied external force would dislodge them from the crypt.

The extraction tip 105 may feature filleted edges 210 to eliminate any sharp or abrupt edges such that all edges are soft and non-damaging to tissue. A smooth filleted edge 210 may minimize potential damage to the tonsil tissue by preventing the tissue from being cut or scraped. According to another aspect of the invention every edge on the device 100 may be filleted so that the entirety of the device 100 is smooth with no rough or sharp edges.

The teardrop shaped 201 aperture 106 in the extraction tip 105 is specialized to not only remove the tonsil stone from the tonsil, but carry the tonsil stone out of the mouth for a significantly improved user experience. After removing the stone from the tonsil, it is preferable for users to also remove the stone from one's mouth. Although tonsil stones are safe to ingest, there is an “ick” factor in doing so, and users most commonly hack and spit the stones out of the mouth. The device 100 described herein addresses this issue by not only removing the stone from the tonsil crypt, but also carrying the stone out of the user's mouth without the need for hacking and spitting, increasing the satisfaction of using the device 100. Water or other chemically compatible solution may be used to rinse the trapped tonsil stone from the extraction tip 105. The trapped tonsil stone may also be removed from the extraction tip 105 by tapping the device 100.

According to another embodiment, the device 100 may feature a handle 104 portion comprising a textured length 401 for producing a non-slip grip. The handle 104 may be part of a unitary structure with the main body, or it may be an additional attachment or overlay. For example, an ergonomic handle 104 may be attached over or on the main body's 101 handle 104 to provide a more comfortable user experience. Such ergonomic handles 104 could be made of a plushy silicone like material. The handle 104 may be attached using a variety of methods such as compression fit, mechanical attachment, or adhesive.

The device 100 may also feature a neck 107 between the extraction tip 105 and handle 104 wherein the neck 107 may be cylindrical or ovular in shape and may connect to the base of the extraction tip 105 at the smallest diameter and gradually taper to match the diameter of the handle 104. Having a defined neck 107 as opposed to the main body 101 or handle 104 connecting to the extraction tip 105 allows for a slender geometry, allowing a user to see both the device 100 and their tonsils. Some people, most notably men, report frustration when using their fingers to express their stones due to the size of their finger both causing gag reflex and blocking the view of the tonsil. The slender tapered neck 107 may improve visibility and may reduce the gag reflex in users. Whereas too small of a device 100 could pose a choking risk, too large of a device 100 may increase the risk for triggering a gag reflex. Although the gag reflex is dependent on the person, it is usually triggered by larger objects. The device 100 described herein has a slender design which may minimize the risk for gaging.

An oral hygiene device 100 according to one or more embodiments herein comprises a main body 101 extending between a handle 104 and an extraction tip 105. An aperture 106 exists in the extraction tip 105 wherein the aperture 106 has a first cross-sectional dimension measuring less than 10 mm. The cross-sectional dimension may be regarded as the first side 202 as described above for a teardrop shaped 201 aperture 106, or it may be regarded as a width, length, radius, diameter, or cross-section of a circular, oval, or other shaped aperture. The device 100 further comprises a handle 104 portion comprising a textured length 401 for producing a non-slip grip and a neck 107 between the extraction tip 105 and handle 104.

The present disclosure comprises multiple devices 100 with varying sizes of extraction tips 105. The extraction tips 105 illustrated in FIG. 3A-3C vary in size from 1 to 3 mm in width 207 or height 211, but sizes from 0.1-10 mm sizes are also contemplated. Each extraction tip 105 is specially designed to take into account the varying sizes of both people's tonsillar crypts and the resulting variation in stone size. The extraction tip 105 is designed to be inserted into the tonsil crypt to gently dislodge, capture, and remove tonsil stones. The user will select the most appropriate sized device 100 and extraction tip 105 to match the size of their tonsil stones and crypts.

The device 100 may have dual ends, such that extraction tips 105 are provided on opposite sides of the device 100. The extraction tips 105 may be of the same or different sizes and shapes according to one or more embodiments described herein. The dual ends providing extraction tips 105 on both sides of the device 100 allow for production of a multi-tool where one device 100 supports multiple sizes of extraction tips 105. The dual ends may be connected by a single solid handle on the main body of the device, or it may have a mechanism to reduce the overall size of the device such as a telescoping or folding mechanism connecting the dual ends.

Suitable materials to manufacture the device 100 include but are not limited to plastics, metals, ceramics, and cellulose materials such as wood or bamboo. The material may also be biodegradable, examples include but are not limited to mycelium, starch, bacteria, soy, cellulose, lignin, or natural fiber based or reinforced plastics. The material may also be a stimuli responsive material, composite, or combination of any of the above-mentioned materials. In one embodiment, the device 100 is constructed from a medical grade material, and for ease of production may be a thermoplastic resin such as polypropylene or polyethylene and/or soft materials such as silicone.

These materials may feature a color either within the material itself or applied to the surface. A preferred color would be in contrast to the color of a tonsil stone, such that the color of the device 100 allows the user to more easily see a captured tonsil stone. For example, a white or yellow colored tonsil stone would be easily seen against a black or blue colored device 100.

Either or both of the main body 101 or the section of the device 100 from the neck 107 to the extraction tip 105 may be comprised of a flexible material, according to the definition defined above. A bending mechanism may be created by the flexibility of the material of device 100, such that the angle or shape of the device 100 may be customized by the user to optimize the tonsil stone extraction experience. A bending mechanism may also be created using a stimuli responsive material such that a user could customize the shape of the device 100 by exposing it to light, temperature, pH, humidity, electricity, magnetism, a combination of, or other stimuli. A bending mechanism may also absorb excess force and bend, rather than transferring the force to the tonsil tissue. This bending mechanism may prevent local tissue damage.

In one or more embodiments, the device 100 may comprise a connection point 402 wherein a wearable device 100 can be attached. In the embodiments illustrated, the connection point 402 is shown as an extruded hole in the base of the main body, opposite the extraction tip 105; however, it may alternatively be a protruding connection point 402. The connection points 402 may optionally connect to a wearable device 100 such as a size-adjustable loop around the wrist, arm, or other bodily extremity which will temporarily attach the device 100 to the user while in use, preventing the device 100 from being dropped into the throat causing choking.

According to an embodiment, an oral hygiene device 100 for removing tonsil stones comprises a main body 101 extending between a handle 104 and an extraction tip 105, wherein one or both of the main body 101 and extraction tip 105 are comprised of a stimuli-responsive material such that a geometry of the device 100 is customizable using one or more stimuli selected from the group consisting of light, temperature, pH, humidity, electricity, and magnetism. An aperture 106 exists in the extraction tip 105 wherein the aperture 106 has a width 207, height 211, or cross-sectional dimension at its widest or longest point that is less than 10 mm.

The device 100 is designed to be portable so that an individual with tonsil stones can conveniently bring the device 100 with them. The device 100 may be stored in a travel bag and used wherever the individual needs them such as in an office bathroom or in a train, plane, spaceship, or car.

The device 100 is designed for use in home or health professional settings. It may be used by the individual with tonsil stones, or someone else such as a friend, relative, or caretaker in an at-home setting; or by a health professional such as but not limited to a doctor, nurse, dentist, or oral hygienist.

When used by the individual with tonsil stones, the individual may use a mirror and light for visibility into the throat. The setup most convenient will likely be a bathroom. Most bathrooms have a mirror, sink, and light source. The user can use an additional light source such as a suction-cup light, flash light, makeup-mirror light, or magnifying-mirror light to visualize the tonsils. The user will identify a tonsil stone, then use the device 100 to gently dislodge and remove the stone. The tonsil stone(s) can be rinsed from the device 100 using the sink. After use, the device 100 may be washed with soap and water or other appropriate oral hygiene cleaning system. The device 100 may be stored with oral hygiene supplies until they are next used. The device 100 may also be disposed of between uses, composted, or recycled, depending on the material.

When used in a health professional setting, the device 100 may be used to remove tonsil stones in a single patient and then disposed of or recycled. The device 100 may be re-used according to the protocols of the health professional setting. Some examples of situations where a professional may remove a patient's tonsil stones include in a general health appointment where the patients asks about their tonsil stones; during a dental appointment where tonsil stones are either visible on an X-ray, made obvious by smell or appearance, or requested by a patient; at an urgent care appointment where patient discomfort is due to a tonsil stone; or at an Ear Nose and Throat specialist where the patient is seeking consultation about tonsil stones. Alternatively, the health care professional may not remove the tonsil stone themselves, but provide or recommend the devices 100 to the patient to use at home. The health care professional may demonstrate to the patient the best practices for removal.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. An oral hygiene device for removing tonsil stones comprising:
a main body wherein the main body includes a first end and second end wherein the first end includes a handle and wherein second end includes an extraction tip and wherein the main body has a flexible length extending between the first end and the second end, wherein the flexible length comprises a material providing sufficient flexibility to the flexible length such that the flexible length is configured to bend at an angle with respect to the handle when pressure is applied to the extraction tip;
an aperture in the extraction tip wherein the aperture is configured to hold a tonsil stone wherein the aperture is teardrop shaped and wherein the aperture has a first side measuring less than 10 mm;
a handle portion comprising a textured length for producing a non-slip grip; and
a neck between the extraction tip and handle wherein the neck is cylindrical or ovular in shape and connects to the base of the extraction tip at a smallest diameter of the neck and gradually tapers to match a diameter of the handle.

2. An oral hygiene device comprising:
a main body extending between a handle and an extraction tip, the extraction tip having curved exterior surfaces;
a teardrop shaped aperture in the extraction tip wherein the aperture has a first side that is an arcuate length and a second side this is an arcuate length, where the first and second sides are spaced apart by a platform extending therebetween to form the teardrop shaped aperture having a first cross-sectional dimension measuring less than 10 mm and having a depth sufficient to form the platform within the aperture of the extraction tip, and the platform terminates on one end with a curved surface connecting to the first side and terminates on a second opposing end with a curved surface connecting to the second sides such that the tear drop shaped aperture and platform are configured to expose and trap a tonsil stone therein such that the tonsil stone can be held in place on the platform and carried by the aperture;
a handle portion; and
a neck between the extraction tip and handle portion.

3. The device of claim 2 wherein the device is configured for removal of tonsil stones.

4. The device of claim 2 wherein the main body is comprised of a flexible material, a portion extending from the neck to the extraction tip is comprised of a flexible material, or both.

5. The device of claim 4 wherein the main body, the portion extending from the neck to the extraction tip, or both is comprised of the flexible material and thus holds its shape when it is manually bent.

6. The device of claim 2 wherein the device is a monolithic unitary structure.

7. (canceled)

8. The extraction tip aperture of claim 2 wherein the first and second sides are of equal length.

9. The extraction tip aperture of claim 2 wherein an apex of the teardrop points towards the main body of the device.

10. The extraction tip of claim 2 wherein the extraction tip has size of up to 10 mm.

11. The aperture in accordance with claim 2 wherein a ratio between the radius of the arcuate length of the teardrop shape to the height of the aperture from the apex of the teardrop shape to the intersection of the height with the radius is in the range of 1:0.25 to 1:10.

12. (canceled)

13. The aperture in accordance with claim 2 wherein the platform includes internal geometric features for tonsil stone grip via frictional force.

14. The aperture in accordance with claim 2 wherein the sides of the extraction tip compress with applied pressure to trap a tonsil stone via applied force.

15. The extraction tip in accordance to claim 2 wherein all edges have fillets such that all edges are soft and non-damaging to tissue.

16. The device of claim 2 wherein the neck is tapered along its length to connect the handle portion to a base of the extraction tip.

17. The device of claim 2 that further comprises a connection point wherein a wearable device can be attached thereto.

18. The device in accordance with claim 5 wherein the material is comprised of a stimuli-responsive material such that a geometry of the device is customizable.

19. The device of claim 2 wherein there is a first extraction tip on the first end and a second extraction tip on the second end and wherein the first extraction tip and the second extraction tip are the same or different in size or shape.

20. An oral hygiene device with user manipulatable shape for removing tonsil stones comprising:
a main body extending between a handle and an extraction tip, wherein the main body and optionally the extraction tip are comprised of a stimuli-responsive material configured to fix the main body or both of the main body and extraction tip with a custom geometry using one or more stimuli selected from the group consisting of light, temperature, pH, humidity, electricity, and magnetism;
an aperture in the extraction tip wherein the aperture has a width, height, or cross-sectional dimension at its widest or longest point that is less than 10 mm, and
wherein the oral hygiene device is configured for the user to manipulate the shape of the device to retain a customized bent shape for accessing a tonsil crypt of the user for removing tonsil stones.
